(19) United States
(12) Patent Application Publication
(10) Pub. No.: US 2011/0178412 A1
(43) Pub. Date: Jul. 21, 2011

(54) CANCEROUS OR PRE-CANCEROUS TISSUE VISUALIZATION METHOD AND DEVICE

(75) Inventor: Pierre Orlewski, Ettelbruck (LU)

(73) Assignee: Centre de Recherche Public Gabriel Lippmann, Belvaux (LU)

(21) Appl. No.: 13/009,703

(22) Filed: Jan. 19, 2011

(30) Foreign Application Priority Data
Jan. 21, 2010 (LU) 91 641

(51) Int. Cl. A61B 6/00

(52) U.S. Cl. 600/477

(57) ABSTRACT

The cancerous or pre-cancerous tissue visualization method includes providing a fluorescence intensity image and a fluorescence lifetime image of the tissue;
identifying in the fluorescence lifetime image first pixels corresponding to a region of healthy tissue and second pixels corresponding to a region of cancerous or pre-cancerous tissue;
defining a weighting function mapping fluorescence lifetime values onto weighting factors in a range from a low weighting factor to a high weighting factor such that the weighting function maps a lifetime value contained in the first pixels onto the low weighting factor and a lifetime value contained in the second pixels onto the high weighting factor;
producing a weighted fluorescence intensity image by weighting the intensity values contained in the pixels of the fluorescence intensity image with weighting factors obtained by evaluation of the weighting function at the lifetime values contained in corresponding pixels of the fluorescence lifetime image.

### RELATED APPLICATIONS

This application claims priority to Luxembourg Patent Application Number 91 641 that was filed on Jan. 21, 2010. The entire content of this application is hereby incorporated herein by reference.

### TECHNICAL FIELD

The present invention relates to the field of medical imaging, more particularly to the imaging and visualizing of cancerous or pre-malignant tissue.

### TECHNICAL BACKGROUND

Photodynamic Diagnosis (PD) (or Fluorescence Diagnosis (FD)) is a low-invasive method for optical biopsy of the tissue. Prior to PD, the tissue area under investigation is sensitized by exogenous, externally applied photosensitizer, most usually applied under the form of ointment containing δ-aminolevulinic acid (5-ALA) or one of its derivatives. 5-ALA is one of the precursors of protoporphyrin IX (PpIX), an endogenous fluorophore intervening in heme synthesis. It is known that external application of 5-ALA will lead to excess of endogenously produced PpIX with regard to its normal (low) concentration in the tissue. Due to different physiological, morphological and biochemical factors among which the high vesicularizsation and different enzymatic activity are the dominant, PpIX will rather accumulate in pre-malignant and in tumour cells than in normal, healthy tissue. It maybe relatively easily visualized, just by illuminating investigated area with blue light from so-called Soret band (˜370-408 nm), which excites PpIX and causes it to fluoresce with fluorescence peaks at 630 and at 710 nm. Therefore, under a Wood's lamp, surface-located non-melanoma tumors and pre-malignancies will appear as reddish areas.

Today, PD yields only qualitative information. Its major drawbacks are that a) it is not possible to solve the fluorescence spectra only in frequency domain due to contribution from different fluorophores, b) the exciting light from Soret band does not penetrate deep enough into tissue (which raises problems with size estimation e.g. of nodular basal cell carcinoma (BCC)), and c) the (contrast) ratio between the fluorescence intensities of “pathological” and “normal” tissue is not constant. Indeed, this ratio is strongly affected by a large number of endogenous or external detection factors. For example, the achievable contrast strongly depends on the precise chemical type of prodrug (photosensitizer) employed. In the case of basal cell carcinoma, the ratio expected for 5-ALA is 2:1, whereas methyl ester of 5-ALA (methyl-ALA) may yield a ratio of 9:1 [C. Fritsch et col., Photochem. Photobiol. 1998; 68(2): 218-221]. Cellular uptake of photosensitizer and the efficiency of its conversion to the endogenous PpIX are among principal factors since the fluorescence intensity strongly depends on the concentration of fluorophore in the tissue. The latter will obviously be influenced by the quantity and concentration of topically applied prodrug and the time between the application and the fluorescence measurement. Other factors influencing on fluorescence intensity are temperature, individual characteristics of the skin and even oxygenation. The morphological sub-types of the tumor will also bias detectable contrast ratio, which may be demonstrated with nodular, infiltrative or multifocal superficial subtypes of the basal cell carcinoma. As PpIX exposed to the light will be progressively subjected to photobleaching (conversion of PpIX to photoproducts), the illumination wavelength, intensity and exposure time are also to be considered. The fluorescence intensity furthermore depends on scattering, viewing angle, detection distance and the characteristics of the detection optics. Finally, the locally varying levels of auto-fluorescence of the tissue will dramatically influence the discrimination contrast.

A conventional PD method uses a Wood's lamp, i.e. a UVA lamp the emission of which is filtered down to the 320-400 nm range and usually peaks at 350-360 nm. Band-pass filtering is typically achieved using a so-called Wood's glass—a barium-sodium-silicate glass stained with nickel oxide. A Wood's lamp is used in diagnosis of some fungal infections of the skin and efficiently reveals the presence of porphyrins. Obviously, the image seen in blue light is “polluted” by fluorescence emitted from other endogenous fluorophores, which results in a poor detection contrast. This is a purely qualitative method pretty useful in the first screening but failing in attempt to more precise delimitation of skin pathologies. The contrast may be so weak than in several cases even a well-trained expert cannot succeed with discrimination of affected skin portions.

An imaging system that uses fluorescence intensity is marketed by Biocam GmbH under the name of DyaDerm. That system employs a 12-bit CCD camera. A xenon light source delivers short, sub-100-ms light flashes in the 370-440 nm range. Two types of images of the area to be examined may be recorded: a fluorescence intensity image and a normal colour image. The images may be overlaid and represented in false colours to highlight the cancerous or pre-cancerous tissue. Using 5-ALA photosensitizer and the DyaDerm camera system, T. Gambichler et coll. [Photodermatology, Photoimunology and Photomedicine, 2008, 24, 67-71] set red/green fluorescence (i.e. autofluorescence) contrast ratio to 1.37 and employed this as the only discrimination feature for basal cell carcinoma (BCC) detection and delimitation. Despite reaching a good correlation of the BCC detection, they observed that the estimated mean tumor area was found significantly smaller (97.9 versus 124.5 mm²) than as per clinical examination.

### SUMMARY

It is an object of the present invention to provide for improved visualization of cancerous or pre-cancerous tissue. This object is achieved by a method as claimed in claim 1.

According to the invention, the cancerous or pre-malignant tissue visualization method comprises the following steps:
illuminating the tissue to be examined with excitation radiation (it is assumed that the area under investigation comprises healthy tissue and cancerous or pre-cancerous tissue);
detecting fluorescence light emitted by the excited tissue in response to the illumination,
providing a fluorescence intensity image of the tissue, the fluorescence intensity image comprising a first array of pixels, each of which contains a fluorescence intensity value;
providing a fluorescence lifetime image of the tissue, the fluorescence lifetime image comprising a second array of pixels, each of which contains a fluorescence lifetime value;
localizing a region of healthy tissue and a region of cancerous or pre-malignant tissue in the fluorescence intensity image;
identifying in the fluorescence lifetime image one or more first pixels corresponding to the region of healthy tissue and one or more second pixels corresponding to the region of cancerous or pre-malignant tissue;
defining a (preferably monotonous) weighting function mapping fluorescence lifetime values onto weighting factors in a range from a predetermined low weighting factor to a predetermined high weighting factor based upon the constraint that the weighting function maps a fluorescence lifetime value contained in the one or more first pixels onto the low weighting factor and a fluorescence lifetime value contained in the one or more second pixels onto the high weighting factor;
producing a weighted fluorescence intensity image by weighting the fluorescence intensity values contained in the pixels of the fluorescence intensity image with weighting factors obtained by evaluation of the weighting function at the fluorescence lifetime values contained in corresponding pixels of the fluorescence lifetime image;
displaying the weighted fluorescence intensity image.

As will be appreciated, the present method relies not only upon fluorescence intensity to visualize cancerous or pre-malignant tissue but takes into account fluorescence lifetime as well. Fluorescence lifetime is in principle independent from intensity and therefore may serve as an additional discriminator to distinguish healthy from cancerous or pre-malignant tissue. For instance, a single decay fluorescent lifetime of PpIX is substantially higher than lifetimes of other endogenous fluorophores contributing to the observable overall fluorescence. The literature (e.g. K. Konig, J. Biophoton [2008], 1, [1]: 13-23) indicates that deep-tissue PpIX has a fluorescence lifetime (τ) of at least 10 ns, whilst other natural fluorophores and not related to cellular malignancy, excitable at same wavelengths will display much shorter lifetime typically below 3 ns (free and protein-bound NAD(P)H, elastin, collagen) or 5.2 ns (flavines). Therefore, one may assume as a general rule that the higher or lower the measured (overall) fluorescence lifetime is, the higher or lower, respectively, is also the relative concentration of PpIX in the mixture of fluorophores.

It should be noted that the present method not only juxtaposes the fluorescence intensity image and the fluorescence lifetime image but provides an enhanced fluorescence intensity image, wherein the intensity values are weighted according to the corresponding lifetime values. However, separate or juxtaposed displaying of the fluorescence intensity image and the fluorescence lifetime image may be optional steps of the method.

The visualization method may e.g. be used to visualize surface-located non-melanoma skin tumours like Basal cell carcinoma, (BCC) and squamous cell carcinoma (SQC) or pre-malignant dysplasia and neoplasia like Bowens disease (BD) or actinic keratosis. It may also be used in examinations of gynaecological and of lower genital tract pathologies and diseases [cf. Hillemans P. et al. 2008], such as cervical intraepithelial neoplasia (GIN) [cf. Hillemans P, Weingardt H., et al., 2000, Andrejevic-Blant S. et al. 2004; Hillemans P. et al. 2009], vulvar intraepithelial neoplasia (VIN), [cf. Hillemans P. et al., 2009, Nowakowski Z. et al., 2005] and HPV-related condyloma acuminate, [cf. Fehr M K et al., 2002]. When additionally employing an endoscopic or laparoscopic interface, the method may be applied to lower genital tract tumours like ovarian carcinoma metastases [cf. Löning M. et al., 2004], squamous intraepithelial lesion (SIL) of the cervix [cf. Collinot P. et al., 2006]. Finally, carcinoma in situ (CIS) of the bladder [cf. Jocham D. et al., 2008; Jichlinski P. and Jacquemin D., 2008; Cauberg E. et al. 2009], other cancers like prostate cancer, penile carcinoma, kidney tumours and urethral condyloma may be visualized using this method. The method may also be used to provide visual feedback in the context of microsurgery of brain malignant gliomas [cf. Stummer W. et al., 2006].

Preferably, the tissue is illuminated with intensity-modulated (i.e. pulsed) excitation radiation so that the fluorescence light fluoresced by the illuminated tissue is also intensity-modulated. The fluorescence lifetime image may thus be recorded with an imager capable of detecting the phase shift between the modulation of the fluorescence light and the modulation of the excitation radiation. Such phase shift (Φ) indicates fluorescence lifetime (τ) via the relationship:

τ=tan(Φ)(2πω)

where ω represents the modulation frequency. The fluorescence lifetime is preferably measured using a so-called lock-in imager, such as described, for instance in the paper “All Solid-State Lock-In Imaging for Wide-Field Fluorescence Lifetime Sensing”, by Esposito A. et al., Optics Express 9812, Vol. 13, No. 24, which is herewith incorporated herein by reference in its entirety for those countries in which the legislation permits such incorporation by reference.

The localization of a region of healthy tissue and a region of cancerous or pre-malignant tissue may comprise displaying the fluorescence intensity image, and receiving localization information on the region of healthy tissue and region of cancerous or pre-cancerous tissue via user interaction. In other words, it may be left to the user to select the regions of healthy and cancerous or pre-cancerous tissue in the fluorescence intensity image that shall serve as a basis for the calculation of the weighting function. In this case, the user may be prompted to select regions, which he is sure are healthy and cancerous or pre-cancerous, respectively. Alternatively, the localization of a region of healthy tissue and a region of cancerous or pre-malignant tissue in the fluorescence intensity image may be carried out in an automated fashion based upon the fluorescence intensity values contained in the pixels of the fluorescence intensity image. For instance, the region displaying the highest fluorescence intensity values could be chosen as the region of cancerous or pre-malignant tissue. Similarly—if necessary after identification and discarding of deemed necrotic regions—the region displaying the lowest fluorescence intensity values could be chosen as the region of healthy tissue. As those skilled will appreciate, the regions of healthy tissue and cancerous or pre-cancerous tissue, respectively, serve the purpose of calibration of the weighting function. It may be noted, with respect to the case where user selects these calibration regions, that the user normally knows at least one spot of cancerous or pre-malignant tissue (what is unknown to him is typically the exact boundary of that tissue) and at least one spot of healthy tissue (typically at some safe distance from the cancerous or pre-cancerous spot).

Preferably, the step of detecting the fluorescence light comprises providing the fluorescence intensity image of the tissue by recording it with a CMOS or CCD imager. Such CMOS or CCD imager should preferably be optimized for detecting the fluorescence wavelengths of PpIX. Ideally, the imager should have a quantum efficiency of above 50% in the wavelength range 600-700 nm and/or a dynamic range of at least 8 bit (more preferably at least 12 bit). The step of detecting the fluorescence light preferably also comprises providing the fluorescence lifetime image of the tissue by recording it with a (e.g. CMOS/CCD) lock-in imager synchronized with the excitation radiation.

More preferably, detecting the fluorescence light comprises recording a plurality of primary fluorescence intensity images of the tissue with a CMOS or CCD imager, the fluorescence intensity image (i.e. the one from which the weighted fluorescence intensity image is produced) being computed from a combination of the primary fluorescence intensity images. The plurality of primary fluorescence intensity images could e.g. be recorded when the tissue is illuminated with excitation radiation at different wavelengths and/or by using different filters to filter the fluorescence light. Detecting the fluorescence light may also comprise recording a plurality of primary fluorescence lifetime images of the tissue with a lock-in imager synchronized with the excitation radiation, the fluorescence lifetime image being computed from a combination of the primary fluorescence lifetime images. The plurality of primary fluorescence lifetime images could e.g. be recorded when the tissue is illuminated with excitation radiation at different wavelengths and/or by using different filters to filter the fluorescence light. It should be noted that the terms “primary fluorescence intensity image” and “primary fluorescence lifetime image” are used here to designate images as recorded by the respective detector, prior to any image processing like contrast enhancement, combination of two or more primary images etc., resulting in one ore more “processed images”. Nevertheless, the primary images and the processed images are preferably of the same type, such as e.g. bitmap.

If separate imagers are used to provide the fluorescence intensity images and the fluorescence lifetime images, respectively, the fluorescence intensity image may have a higher imager resolution than the fluorescence lifetime image. This means that one spot in the imaged area may be imaged onto a higher number of pixels by the CMOS or CCD imager than by the lock-in imager. In this case, there is no one-to-one correspondence between the pixels of the intensity image and those of the lifetime image, which one would ideally have in the case of identical image resolutions and identical imaged areas. Accordingly the producing of a weighted fluorescence intensity image comprises: for each pixel of the fluorescence intensity image, determining a corresponding pixel in the fluorescence lifetime image, evaluating the weighting function at the fluorescence lifetime value contained in the determined corresponding pixel to find a weighting factor and weighting the fluorescence intensity value contained in the pixel of the fluorescence intensity image with the found weighting factor.

Preferably, the visualization method according to the invention comprises applying ointment containing a protoporphyrin IX precursor, e.g. δ-aminolevulinic acid or a derivative thereof (e.g. methyl ALA), onto the tissue.

An aspect of the invention concerns a medical imaging device configured to visualize cancerous or pre-cancerous tissue according to the aforementioned method. Such imaging device comprises
an illumination unit configured to emit excitation radiation onto tissue to be imaged;
a CCD or CMOS imager for recording a fluorescence intensity image of the tissue to be imaged, the fluorescence intensity image comprising a first array of pixels, each of which contains a fluorescence intensity value;
a (CMOS/CCD) lock-in imager for recording a fluorescence lifetime image of the tissue to be imaged, the fluorescence lifetime image comprising a second array of pixels, each of which contains a fluorescence lifetime value;
a processor connected to the CCD or CMOS imager and to the CMOS/CCD lock-in imager, the processor being configured
   to localize (autonomously or via interaction with a user) a region of healthy tissue and a region of cancerous or pre-cancerous tissue in the fluorescence intensity image,
   to identify in the fluorescence lifetime image one or more first pixels corresponding to the region of healthy tissue and one or more second pixels corresponding to the region of cancerous or pre-malignant tissue,
   to define a (preferably monotonous) weighting function mapping fluorescence lifetime values onto weighting factors in a range from a predetermined low weighting factor to a predetermined high weighting factor based upon the constraint that the weighting function maps a fluorescence lifetime value contained in the one or more first pixels onto the low weighting factor and a fluorescence lifetime value contained in the one or more second pixels onto the high weighting factor and
   to produce a weighted fluorescence intensity image by weighting the fluorescence intensity values contained in the pixels of the fluorescence intensity image with weighting factors obtained by evaluation of the weighting function at the fluorescence lifetime values contained in corresponding pixels of the fluorescence lifetime image; and
a display connected to the processor to display the weighted fluorescence intensity image.

### BRIEF DESCRIPTION OF THE DRAWINGS

Further details and advantages of the present invention will be apparent from the following detailed description of not limiting embodiments with reference to the attached drawings, wherein:

FIG. 1 is a schematic view of a medical imager for cancerous or pre-cancerous tissue visualization;

FIG. 2 is a flow chart illustrating a cancerous or pre-cancerous tissue visualization method according to one or more embodiments;

FIG. 3 is a flow chart illustrating a variant of the method of FIG. 2; and

FIG. 4 shows graphs of weighting functions as may be used in various embodiments;

### DETAILED DESCRIPTION

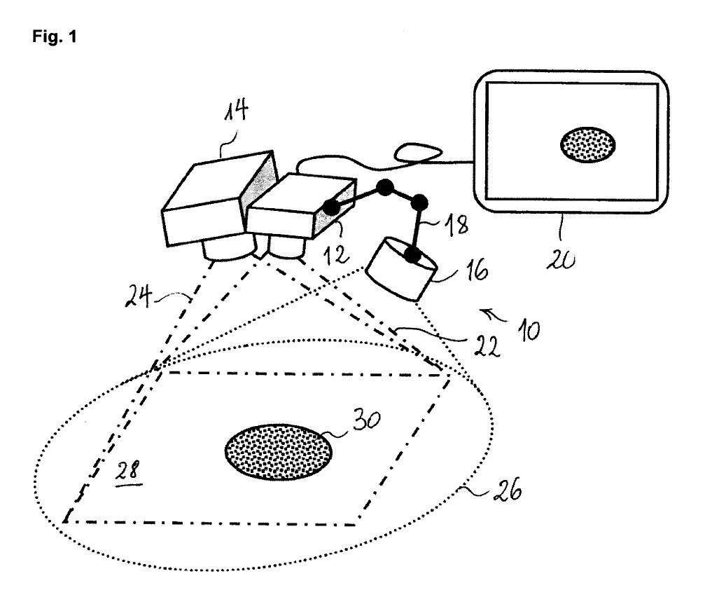

A medical imaging device 10 for cancerous or pre-cancerous tissue visualization is shown in FIG. 1. The imaging device 10 comprises a hand-held unit with a CCD or CMOS camera 12 for recording fluorescence intensity images and a CCD/CMOS lock-in imager 14 for detecting fluorescence lifetime images. The CCD or CMOS camera 12 and the CCD/CMOS lock-in imager 14 are arranged in such a way that their respective fields of view 22, 24 substantially overlay and coincide. The imaging device 10 further comprises an illumination unit 16, which is fixed to the housing of the CCD or CMOS camera 12 and the CCD/CMOS lock-in imager 14 with a hinged joint 18, a processor (not shown, integrated into the housing of the imagers 12, 14) and a display device 20.

The CCD or CMOS camera 12 exhibits a resolution of at least 1200×800 pixels, a quantum efficiency of above 50% in the 600-700 nm range and 12 bit or higher dynamic range. The CCD/CMOS lock-in imager 14 typically exhibits substantially lower resolution than the CCD or CMOS camera 12. For technical reasons, the resolution of the CCD or CMOS camera 12 is limited today to maximum 300×200 pixels with a quantum efficiency above 60% in similar radiation range.

Both imagers 12 and 14 are individually equipped with dedicated optics, ensuring similar effective fields of view. They are furthermore equipped with filters to selectively block the illumination light. The filters may be changed manually or automatically by means e.g. of a motorised filter wheel or the like.

The illumination unit 16 is controlled to emit intensity-modulated (pulsed) light at least in the Soret band that corresponds to the maximum absorption of PpIX (i.e. at about 403 nm). The CCD/CMOS lock-in imager 14 is synchronized with the illumination unit and detects the phase shift between the modulation of the emitted fluorescence light and the modulation of the excitation light. The phase detection principle used by the imager is the same as the one employed in so-called 3D time-of-flight cameras and is explained in the aforementioned paper by Esposito et al. For each pixel, the lock-in imager 14 thus measures a phase shift (Φ) from which the fluorescence lifetime (τ) may be computed as τ=tan(Φ)/(2πω)). For improved synchronism of the CCD/CMOS lock-in imager 14 with the illumination unit, some of the pixels of the lock-in imager may be directly coupled via a light guide to the illumination unit and shielded against the light coming from the imaged scene. These pixels thus provide a reference phase with respect to which the phase of the fluorescence light may be calculated.

The excitation light pulses of the illumination unit are generated by solid-state, semiconducting light emitters like LEDs or VCELS. Their number is selected in the way to reach at least a predefined fluence, selected preferably in the range from 0.5 to 15 mW/cm² and optimally at 2.0 mW/cm² at skin plane. The LEDs or VCELS are distributed in such a way as to provide a homogenous distribution of the light at detection plane. To achieve this as well as to adjust the size of the illuminated area 26, a light shaping collimator may be arranged on top of the LEDs or VCSELs. The modulation frequency is preferably selected in the frequency range from 13 kHz to 40 MHz.

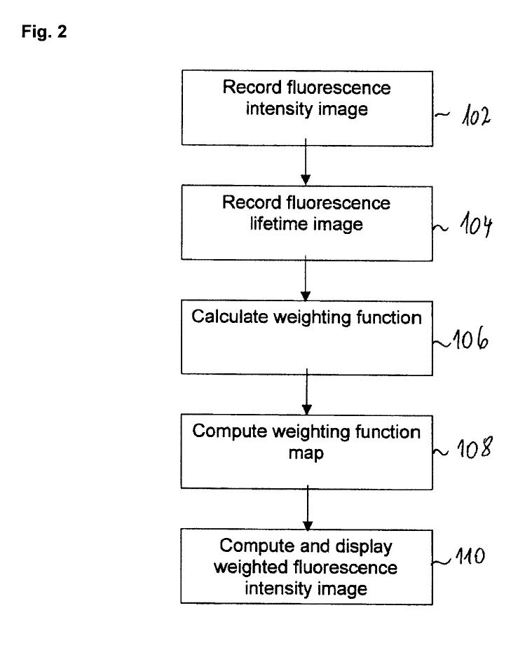

A preferred embodiment of the method according to which the device of FIG. 1 may operate is illustrated in FIG. 2. After the cancerous or pre-cancerous tissue has been found by conventional, optical inspection at ambient light or under a Wood's lamp, photosensitizer (e.g. 5-ALA) is applied to the area to be examined. After the prescribed time has lapsed, the tissue to be examined is illuminated with excitation radiation from the illumination unit 16 and the fluorescence light is detected by means of the imagers 12 and 14. The CCD or CMOS camera 12 records a fluorescence intensity image of the tissue (step 102), while the lock-in imager 14 records a fluorescence lifetime image of the tissue (step 104).

In the next step 106, the processor (which may e.g. be an application-specific integrated circuit, a field-programmable gate array, a digital signal processor or the like) now localizes a region 28 of healthy tissue and a region 30 of cancerous or pre-cancerous tissue in the fluorescence intensity image. It may e.g. achieve this by first eliminating those regions from consideration, in which the fluorescence intensity is locally abnormally low or even null (indicating necrotic tissue), and then setting those pixels with the highest intensity values and those pixels with the lowest intensity values as the regions of cancerous or pre-cancerous tissue and the region of healthy tissue, respectively. The processor then identifies one or more first pixels corresponding to the region of healthy tissue and one or more second pixels corresponding to the region of cancerous or pre-cancerous tissue in the fluorescence lifetime image. In the fluorescence intensity image and the fluorescence lifetime image, “corresponding” pixels are pixels containing information about the same imaged spot. The correspondence could be a one-to-one correspondence in case of identical image resolutions and perfect superposition of the fields of view. The general case is, however, that there is at least a parallax error between the two images and additionally also a difference in the image resolutions. The processor further defines a weighting function mapping fluorescence lifetime values onto weighting factors in a range from a predetermined low weighting factor (e.g. 0) to a predetermined high weighting factor (e.g. 1) based upon the constraint that the weighting function maps a fluorescence lifetime value contained in the one or more first pixels onto the low weighting factor and a fluorescence lifetime value contained in the one or more second pixels onto the high weighting factor. Graphs of possible weighting functions are shown in FIG. 4. The dashed graph corresponds to a linear weighting function (given by y(τ)=(τ−τₘᵢₙ)/(τₘₐₓ−τₘᵢₙ)), whereas the dotted graph corresponds to a monotonously growing non-linear weighting function.

The processor then generates a map (having the image resolution of the fluorescence intensity image) wherein each pixel contains the weighting factor obtained by evaluation of the weighting function at the fluorescence lifetime values contained in corresponding pixels of the fluorescence lifetime image (step 108).

A weighted fluorescence intensity image is then computed by weighting (multiplying) the fluorescence intensity values contained in the pixels of the fluorescence intensity image with the weighting factors (ranging from 0 to 1) contained in the corresponding pixels of the weighting map.

The so-obtained weighted fluorescence intensity image is finally displayed on display 20 (step 110).

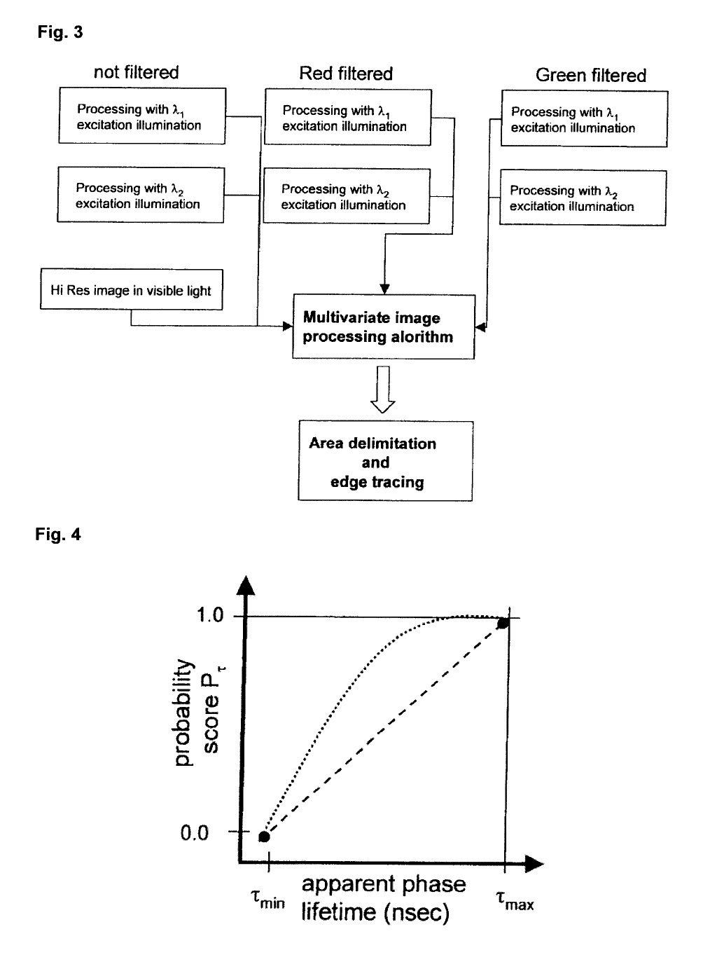

According to a preferred embodiment of the method (schematically illustrated in FIG. 3), several fluorescence intensity and lifetime images are recorded. During a first period, the tissue to be examined is illuminated with excitation radiation at a first wavelength (selected in the range from 370 to 430 nm, preferably at about 408 nm). During the first period, three fluorescence intensity images of the tissue are recorded. One of these images is recorded using no filter, another of these images is recorded using a filter having a transmission window from 320 to 550 nm, whereas the third of these images is recorded using a filter having a transmission window from 610 to 750 nm. During a second period, the tissue to be examined is illuminated with excitation radiation at a second wavelength (selected in the range from 500 to 600 nm, preferably at about 530 nm). During the second period, fluorescence intensity and lifetime images are again recorded after delay time T, preferably of few minutes using the abovementioned filters.

The processor combines the primary fluorescence intensity images into a processed fluorescence intensity image. By morphing, warping and/or stretching, all three images are brought to exactly the same reference system. For every image pixel found in the first intensity image (no filter), the radiant background is estimated, for example by fitting pixel intensity slopes with respectively second (320-550 nm) and third (610-750 nm) image pixels.

The processor combines the primary fluorescence lifetime images into a processed fluorescence lifetime image. After subtraction of the radiant background, an artificial image is created based on all images collected during the first and the second period. The processor combines these images in such a way that the intensity of every pixel of the artificial image is given as linear or second order combination of intensities from all collected images. In the following, A1 designates the (relative) weight of the image recorded during the first period without a filter, B1 designates the (relative) weight of the image recorded during the first period with a filter having a transmission window from 320 to 550 nm and C1 designates the (relative) weight of the image recorded during the first period with a filter having a transmission window from 610 to 750 nm. A2 designates the (relative) weight of the image recorded during the second period (after delay T) without a filter, B2 designates the (relative) weight of the image recorded during the second period with a filter having a transmission window from 320 to 550 nm and C2 designates the (relative) weight of the image recorded during the second period with a filter having a transmission window from 610 to 750 nm. The constant parameters A1, B1, C1, A2, B2 and C2 of this combination may be selected in such a way that the combination raises those intensities collected at higher wavelengths (for example by setting A1=1, B1=1.5 and C=2). Such parameter selection takes into account the fact that the higher is the fluorescence wavelength, the bigger is the probability that the measured light intensity originates from PpIX and not from another chromophore present in the skin and not related to potential skin malignancy. Alternatively or additionally, the constant parameters may be selected to account for the change (reduction) of the intensities of the images within the same filter configuration (without filter, filter with transmission window from 320 to 550 nm and filter with a transmission window from 610 to 750 nm, respectively) but collected during the first period and the second period, separated by the delay time T. Indeed, the higher is the pixel intensity loss observed (due to fluorophore photobleaching) from the first to the second period, the bigger is the likelihood that the light emission came from malignant tissue (because the concentration of PpIX fluorophore is higher in such tissue, which furthermore lies relatively close to the skin surface and is, therefore, more exposed to the incident light). Preferably, the 3×2 matrix of coefficients A1, B1, C1, A2, B2, and C2 is set and validated experimentally.

The processor now localizes a region of healthy tissue and a region of cancerous or pre-cancerous tissue in the processed fluorescence intensity image. It may e.g. achieve this by first eliminating those regions from consideration, in which the processed fluorescence intensity is locally abnormally low (indicating necrotic tissue), and then setting those pixels with the highest processed intensity values and those pixels with the lowest processed intensity values as the regions of cancerous or pre-cancerous tissue and the region of healthy tissue, respectively. In the next step, the processor identifies one or more first pixels corresponding to the region of healthy tissue and one or more second pixels corresponding to the region of cancerous or pre-cancerous tissue in the processed fluorescence lifetime image.

The processor defines a weighting function mapping processed fluorescence lifetime values onto weighting factors in a range from a predetermined low weighting factor (here: 0) to a predetermined high weighting factor (here: 1) based upon the constraint that the weighting function maps a processed fluorescence lifetime value contained in the one or more first pixels onto the low weighting factor and a processed fluorescence lifetime value contained in the one or more second pixels onto the high weighting factor. The processor then generates a map (having the image resolution of the fluorescence intensity images) wherein each pixel contains the weighting factor obtained by evaluation of the weighting function at the processed fluorescence lifetime values contained in corresponding pixels of the processed fluorescence lifetime image.

A weighted fluorescence intensity image is then computed by weighting (multiplying) the processed fluorescence intensity values contained in the pixels of the fluorescence intensity image with the weighting factors (ranging from 0 to 1) contained in the corresponding pixels of the weighting map.

The so-obtained weighted fluorescence intensity image is finally displayed on display 20.

The invention has been described with reference to various specific and preferred embodiments and techniques. However, it should be understood that many variations and modifications may be made while remaining within the spirit and scope of the invention.

What is claimed is:

1. A cancerous or pre-cancerous tissue visualization method, comprising
providing a fluorescence intensity image of a tissue comprising healthy tissue and cancerous or pre-cancerous tissue, said fluorescence intensity image comprising a first array of pixels, each of which contains a fluorescence intensity value;
providing a fluorescence lifetime image of said tissue, said fluorescence lifetime image comprising a second array of pixels, each of which contains a fluorescence lifetime value;
localizing a region of healthy tissue and a region of cancerous or pre-cancerous tissue in said fluorescence intensity image;
identifying in said fluorescence lifetime image one or more first pixels corresponding to said region of healthy tissue and one or more second pixels corresponding to said region of cancerous or pre-cancerous tissue;
defining a weighting function mapping fluorescence lifetime values onto weighting factors in a range from a predetermined low weighting factor to a predetermined high weighting factor based upon the constraint that said weighting function maps a fluorescence lifetime value contained in the one or more first pixels onto said low weighting factor and a fluorescence lifetime value contained in the one or more second pixels onto said high weighting factor;
producing a weighted fluorescence intensity image by weighting said fluorescence intensity values contained in the pixels of said fluorescence intensity image with weighting factors obtained by evaluation of said weighting function at the fluorescence lifetime values contained in corresponding pixels of said fluorescence lifetime image; and
displaying said weighted fluorescence intensity image.

2. The visualization method as claimed in claim 1, wherein prior to providing said fluorescence intensity image and said fluorescence lifetime image, said method comprises illuminating said tissue with excitation radiation and detecting fluorescence light emitted by said illuminated tissue in response to said illumination.

3. The visualization method according to claim 2, wherein said tissue is illuminated with intensity-modulated excitation radiation so that said fluorescence light fluoresced by said illuminated tissue is also intensity-modulated and wherein said fluorescence lifetime image is recorded with an imager capable of detecting a phase shift between the modulation of the fluorescence light and the modulation of the excitation radiation, said phase shift being indicative of fluorescence lifetime.

4. The visualization method according to claim 1, wherein said localizing of a region of healthy tissue and a region of cancerous or pre-cancerous tissue comprises displaying said fluorescence intensity image, and receiving localization information on said region of healthy tissue and region of cancerous or pre-cancerous tissue via user interaction.

5. The visualization method according to claim 1, wherein said localizing of a region of healthy tissue and a region of cancerous or pre-cancerous tissue in said fluorescence intensity image based upon the fluorescence intensity values contained in the pixels is carried out in an automated fashion based upon the fluorescence intensity values contained in the pixels of said fluorescence intensity image.

6. The visualization method according to claim 2, wherein detecting said fluorescence light comprises providing said fluorescence intensity image of said tissue by recording it with a CMOS or CCD imager, and wherein detecting said fluorescence light comprises providing said fluorescence lifetime image of said tissue by recording it with a lock-in imager synchronized with said excitation radiation.

7. The visualization method according to claim 2, wherein detecting said fluorescence light comprises recording a plurality of primary fluorescence intensity images of said tissue with a CMOS or CCD imager, and wherein said fluorescence intensity image is computed from a combination of said primary fluorescence intensity images.

8. The visualization method according to claim 7, wherein said plurality of primary fluorescence intensity images is recorded when said tissue is illuminated with excitation radiation at different wavelengths.

9. The visualization method according to claim 7, wherein said plurality of primary fluorescence intensity images is recorded using different filters to filter the fluorescence light.

10. The visualization method according to claim 2, wherein detecting said fluorescence light comprises recording a plurality of primary fluorescence lifetime images of said tissue with a lock-in imager synchronized with said excitation radiation, and wherein said fluorescence lifetime image is computed from a combination of said primary fluorescence lifetime images.

11. The visualization method according to claim 10, wherein said plurality of primary fluorescence lifetime images is recorded when said tissue is illuminated with excitation radiation at different wavelengths.

12. The visualization method according to claim 10, wherein said plurality of primary fluorescence lifetime images is recorded using different filters to filter the fluorescence light.

13. The visualization method according to claim 1, wherein said fluorescence intensity image has a higher image resolution than said fluorescence lifetime image, and wherein said producing of a weighted fluorescence intensity image comprises: for each pixel of said fluorescence intensity image, determining a corresponding pixel in said fluorescence lifetime image, evaluating said weighting function at the fluorescence lifetime value contained in the determined corresponding pixel to find a weighting factor and weighting the fluorescence intensity value contained in the said pixel of the fluorescence intensity image with the found weighting factor.

14. The visualization method according to claim 1, comprising applying ointment containing a protoporphyrin IX precursor, e.g. δ-aminolevulinic acid or a derivative thereof, onto said tissue.

15. Cancerous or pre-cancerous tissue visualization method, comprising illuminating tissue with excitation radiation, said tissue comprising healthy tissue and cancerous or pre-cancerous tissue;
detecting fluorescence light emitted by said illuminated tissue in response to said illumination,
providing a fluorescence intensity image of said tissue based upon said fluorescence light detected, said fluorescence intensity image comprising a first array of pixels, each of which contains a fluorescence intensity value;
providing a fluorescence lifetime image of said tissue based upon said fluorescence light detected, said fluorescence lifetime image comprising a second array of pixels, each of which contains a fluorescence lifetime value;
identifying in said fluorescence lifetime image one or more first pixels corresponding to a region of healthy tissue and one or more second pixels corresponding to a region of cancerous or pre-cancerous tissue;
defining a weighting function mapping fluorescence lifetime values onto weighting factors in a range from a predetermined low weighting factor to a predetermined high weighting factor based upon the constraint that said weighting function maps a fluorescence lifetime value contained in the one or more first pixels onto said low weighting factor and a fluorescence lifetime value contained in the one or more second pixels onto said high weighting factor;
producing a weighted fluorescence intensity image by weighting said fluorescence intensity values contained in the pixels of said fluorescence intensity image with weighting factors obtained by evaluation of said weighting function at the fluorescence lifetime values contained in corresponding pixels of said fluorescence lifetime image.

16. A medical imaging device configured to visualize cancerous or pre-cancerous tissue, the device comprising
an illumination unit configured to emit excitation radiation tissue to be imaged;
a CCD or CMOS imager for recording a fluorescence intensity image, said fluorescence intensity image comprising a first array of pixels, each of which contains a fluorescence intensity value;
a lock-in imager for recording a fluorescence lifetime image of said tissue, said fluorescence lifetime image comprising a second array of pixels, each of which contains a fluorescence lifetime value;
a processor connected to said CCD or CMOS imager and to said lock-in imager, said processor being configured
to localize a region of healthy tissue and a region of cancerous or pre-cancerous tissue in said fluorescence intensity image,
to identify in said fluorescence lifetime image one or more first pixels corresponding to said region of healthy tissue and one or more second pixels corresponding to said region of cancerous or pre-cancerous tissue,
to define a weighting function mapping fluorescence lifetime values onto weighting factors in a range from a predetermined low weighting factor to a predetermined high weighting factor based upon the constraint that said weighting function maps a fluorescence lifetime value contained in the one or more first pixels onto said low weighting factor and a fluorescence lifetime value contained in the one or more second pixels onto said high weighting factor and
to produce a weighted fluorescence intensity image by weighting said fluorescence intensity values contained in the pixels of said fluorescence intensity image with weighting factors obtained by evaluation of said weighting function at the fluorescence lifetime values contained in corresponding pixels of said fluorescence lifetime image; and
a display connected to said processor to display said weighted fluorescence intensity image.

17. The medical imaging device as claimed in claim 15, comprising an endoscopic or a laparoscopic interface equipped with said illumination unit, said CCD or CMOS imager and said lock-in imager.
